(19) United States
(12) Patent Application Publication
(10) Pub. No.: US 2023/0248710 A1
(43) Pub. Date: Aug. 10, 2023

(54) SYNERGISTIC COMPOSITION FOR VEISALGIA PREVENTION

(71) Applicant: Ajay Perecherla, Madison, WI (US)

(72) Inventor: Ajay Perecherla, Madison, WI (US)

(21) Appl. No.: 17/665,493

(22) Filed: Feb. 5, 2022

(51) Int. Cl. A61K 31/4525; A61K 31/12

(52) U.S. Cl. CPC A61K 31/4525; A61K 31/12

(57) ABSTRACT

A composition of powder mixture is disclosed that reduces the effects of hangovers by preventing inflammation of the brain by using two natural ingredients: curcumin and piperine.

### FIELD OF THE INVENTION

The invention relates generally to an orally consumable composition and, more specifically, to a powder mixture that counteracts the effect of a hangover resulting from alcohol.

### BACKGROUND

A series of physiologic symptoms, commonly referred to as veisalgia (“hangover”), are usually experienced after ingesting alcohol. The term “alcohol” as used herein refers to ethyl alcohol (ethanol) and alcoholic beverages intended for human consumption. Symptoms can include nausea, headache, dizziness, and stomach pain. The cause of several hangover symptoms is attributed to the byproducts formed by the body's inflammatory response to the liver's metabolism of the ingested alcohol. When alcohol is ingested, ethanol converts into acetylaldehyde by the enzyme alcohol dehydrogenase.

During that process, prostaglandin PGE2 trigger the inflammatory response to the central nervous system, mainly the brain. Prostaglandins are lipids, or building blocks of fatty acids, that act like hormones and serve various functions in the human body. There exists in the art purported remedies for treating these hangover symptoms.

The role of prostaglandins in hangover is based studies confirming that alcohol consumption is accompanied by an immune response, and that elevated levels of cytokines in blood and saliva may be related to the presence and severity of the alcohol hangover. Alcohol has an effect on prostaglandin synthesis, which can be counteracted by a prostaglandin inhibitor, such as tolfenamic acid. Therefore, what is needed is a remedy, which is an ingestible product, that aims to treat the causes of these symptoms before being manifest in the body.

### SUMMARY

In accordance with the disclosure of the invention, a powder mixture is disclosed that treats the causes of handovers before manifesting through an ingestible product that is easily transportable and highly flexible and simple to implement.

In accordance with the various aspects and embodiments of the invention, a powder mixture containing is disclosed curcumin and piperine. The mixture can be delivered with a liquid or delivered as a solid in tablet or capsule form. Regardless of the delivery means, the effect of the powder mixture is to reduce the inflammatory response to hangover severity from alcohol.

### BRIEF DESCRIPTION OF THE DRAWINGS

In order to understand the invention more fully, reference is made to the accompanying drawings or figures. The invention is described in accordance with the aspects and embodiments in the following description with reference to the drawings or figures (FIG.), in which like numbers represent the same or similar elements. Understanding that these drawings are not to be considered limitations in the scope of the invention, the presently described aspects and the presently understood best mode of the invention are described with additional detail through use of the accompanying drawings.

FIG. 1A shows a brief process of alcohol metabolism that occurs in the liver in which ethanol is converted to acetylaldehyde, which is then converted to usable energy that is used to fuel the body.

FIG. 1B shows how prostaglandins (a type of phospholipid) are created, which are a group of lipids made at sites of tissue damage or infection and, more specifically, prostaglandin PGE₂ creates an inflammatory response in the brain, kidneys, vascular smooth muscle cell (VSMCs), and platelets.

FIG. 1C shows where the powder mixture targets in order to prevent the inflammatory response from happening in accordance with the various aspects and embodiments of the invention.

FIG. 2 shows a schematic of tablet assembly unit in accordance with an embodiment and various aspects of the invention.

### DETAILED DESCRIPTION

In the following detailed description, reference is made to the accompanying drawings which form a part hereof, and in which is shown by way of illustration specific embodiments in which the disclosure may be practiced. These embodiments are described in sufficient detail to enable those skilled in the art to practice the disclosure, and it is to be understood that other embodiments may be utilized and that mechanical, procedural, and other changes may be made without departing from the spirit and scope of the disclosure(s). The following detailed description is, therefore, not to be taken in a limiting sense, and the scope of the disclosure(s) is defined only by the appended claims, along with the full scope of equivalents to which such claims are entitled.

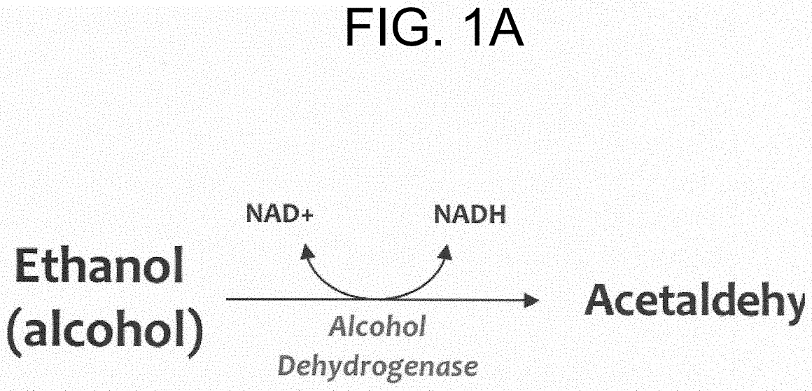

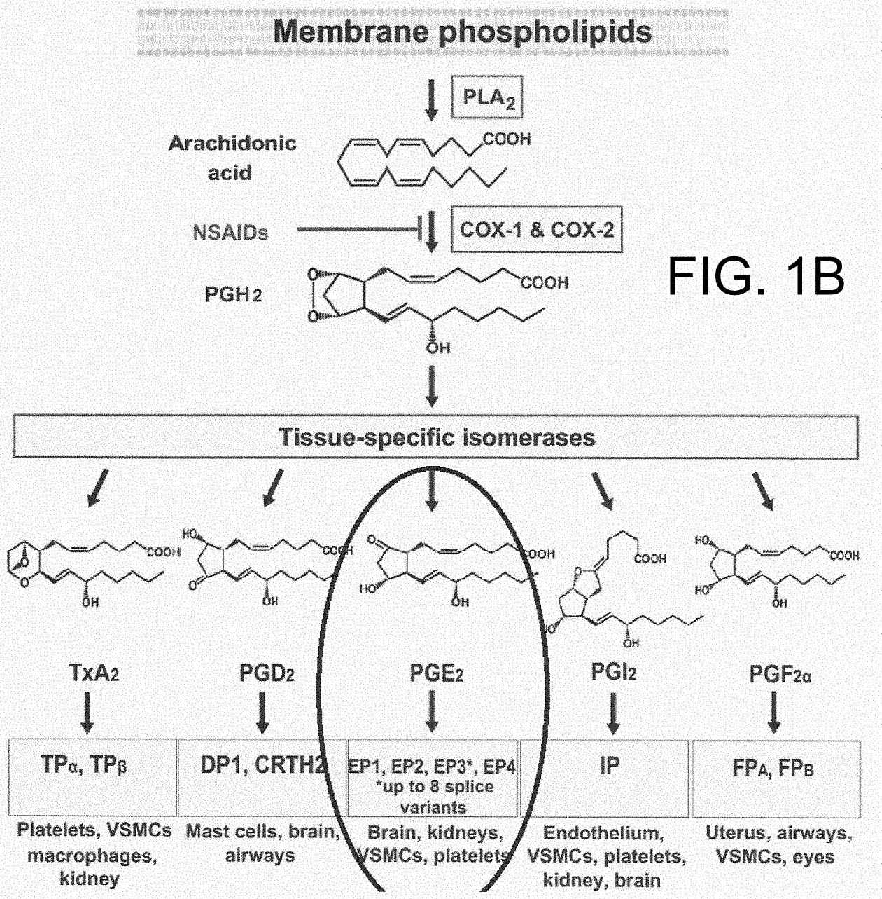

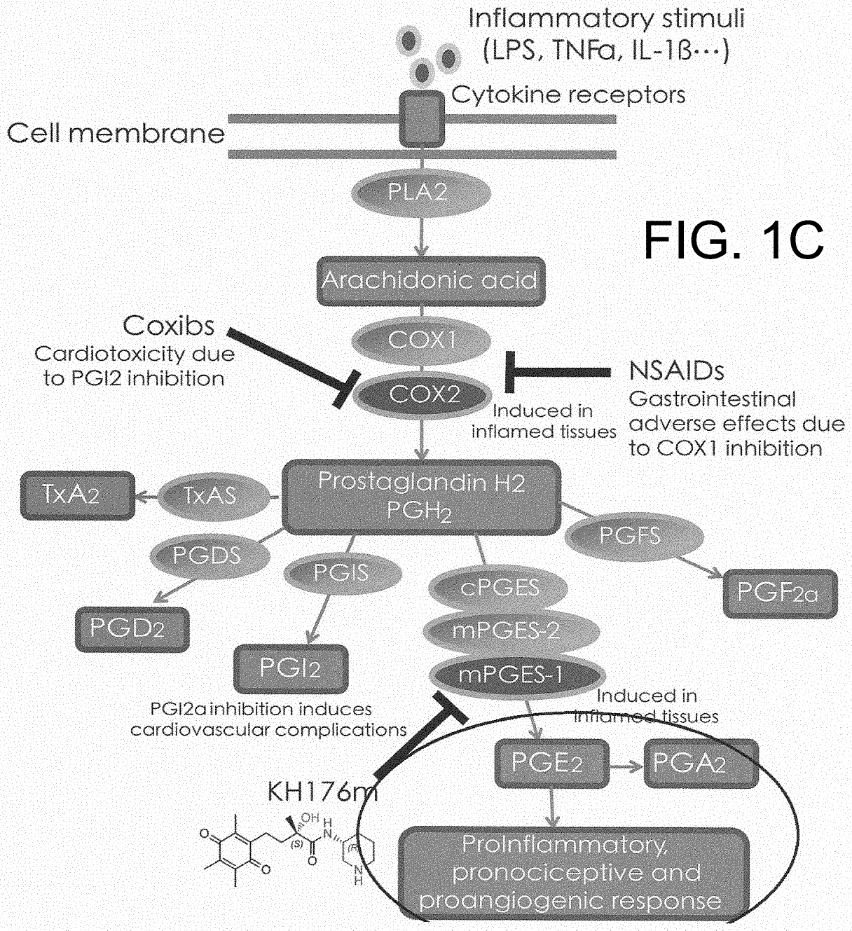

Referring now to FIGS. 1A-1C, various chemical reactions are shown related to the effects of alcohol and the benefit of a hangover prevention mixture. In accordance with various embodiments of the invention, the hangover prevention mixture (powder mixture), both in powder form and in capsule/pill form, is disclosed. The powder mixture can be added to any food or beverage and consumed before, during, or after drinking alcohol to realize its hangover preventing benefits.

The powder mixture is composed of curcumin and piperine. Curcumin is a bright yellow compound found in turmeric and is commonly sold as an herbal supplement boasting anti-inflammatory, anti-tumor, and antioxidant effects on the body. Curcumin combats the deleterious effects of alcohol intoxication through curcumin's anti-inflammatory and antioxidant properties. Piperine is an alkaloid found in black pepper and long pepper. It is used to improve the absorption of curcumin by the human body by increasing its bioavailability. Piperine is used to improve curcumin absorption by the human body, thereby increasing the powder mixture's effectiveness.

In accordance with various embodiments of the invention, there are three variations for delivery that have different doses as the following: the 95% curcumin to piperine powder mixture that is stirred into any liquid, the 98% curcumin to piperine powder mixture that is compressed into a capsule, and the 98% curcumin to piperine powder mixture is compressed into a tablet form.

In accordance with various embodiments of the invention, the 95% curcumin to piperine powder mixture is packaged. For example, the package is a 1.8″×3.5″ shaped packet in a labeled box.

In accordance with various embodiments of the invention, the 98% curcumin to piperine powder mixture is put into a square plastic packet, which is used to package tablets in a labeled box.

In accordance with various embodiments of the invention, the hangover prevention mixture is a powder that is mixed with a liquid (e.g., water) that can be ingested prior to, during, or after consuming alcohol in order to prevent the user from experiencing alcohol induced hangover symptoms. In accordance with various embodiments of the invention, the hangover prevention mixture is in capsule/pill form that can be ingested prior to, during, or after consuming alcohol in order to prevent the user from experiencing alcohol induced hangover symptoms.

In accordance with various embodiments of the invention, a single serving of the hangover prevention mixture includes a ratio of 1.5 grams (g) of curcumin and 0.5 g of piperine, in powder form. In various compositions, the ratio of curcumin:piperine can vary and be above or below the ideal ratio of 3:1. In accordance with various embodiments of the invention, the range for curcumin is 1.0 g-2.0 g and the range for piperine is 0.25 g-1.0 g. In accordance with various embodiments of the invention, the powder mixture includes curcumin that is 95% curcuminoids and in liquid form resulting in a fast release effect. In accordance with various embodiments of the invention, the powder mixture includes curcumin that is 98% curcuminoids and in capsule form resulting in a slow-release effect. In accordance with various embodiments of the invention, the powder mixture includes curcumin that is 98% curcuminoids and in tablet form resulting in a slow-release effect.

In accordance with various embodiments of the invention, each serving of hangover prevention mixture (powder mixture) comes in a packet. The packet is preferably an envelope, wrapper, bag, or any other means for reliably storing and transporting a powdered substance and preserving it from exposure to environment conditions until the packet is opened.

In accordance with various embodiments of the invention, the powder mixture is mixed into a beverage, wherein a beverage refers to any consumable is a largely liquid state, including water. The user can empty the contents of the packet into any beverage of choice of any temperature and volume. The volume of liquid or beverage used is up to the consumer. The user may stir the beverage-powder mixture and wait for the powder to dissolve or consume the mixture right after incorporating the powder mixture. In accordance with various embodiments of the invention, the powder mixture includes or is combined with food. As used herein the term “food” includes any consumable of a largely non-liquid state.

The powder mixture can be consumed at any time. For example, the powder mixture can be consumed 10 to 15 minutes before drinking alcohol or after drinking alcohol. Furthermore, the powder mixture can be consumed while drinking alcohol as well as be mixed into the alcoholic beverage itself. The user needs only to consume a single serving of powder mixture to combat the hangover inducing effects of any volume of alcohol imbibed in a single sitting. In accordance with various embodiments of the invention, the mixture can be fully ingested over any time duration during the course of the 10 to 15 minutes before, during, or the 10 to 15 minutes after alcohol consumption.

In accordance with various embodiments of the invention, the powder mixture includes additives for improving the powder mixture's flavor, consistency, shelf-life, and the like. For example, flavorings can be added to produce a mango flavored or mint flavored powder mixture.

In accordance with various embodiments of the invention, the powder mixture is compressed into whole tablets of any shape or size. One advantage of the tablet form is the slow-release effect. The tablets are packaged into a bottle as outline herein as part of the bottling process.

In accordance with various embodiments of the invention, the powder mixture includes curcumin (98% curcuminoid) and piperine compressed into capsules for the slow releasing effect. The capsule includes a membrane that dissolves to allow the slow release of the powder mixture. In accordance with various embodiments of the invention, the total effect of the powder mixture is release in a time period ranging from 15 minutes to 60 minutes. One advantage of the capsule form is the slower release of the powder mixture, in part due to the membrane of the capsule. Another advantage is prevention of the bitter taste when one consumes the powder mixture. The capsules are packaged into a bottle.

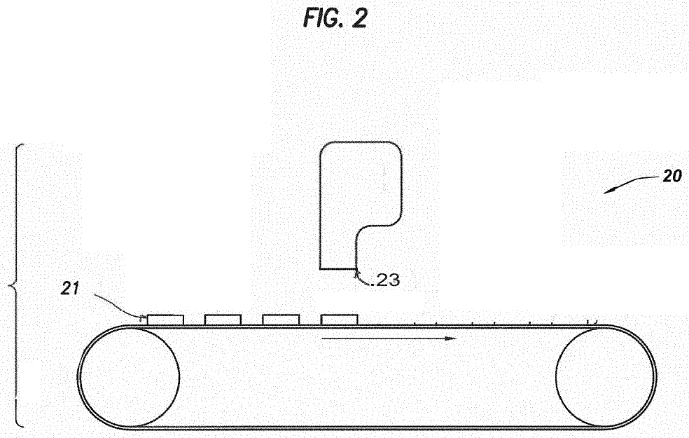

Referring now to FIG. 2, assembly unit 20 is shown in accordance with various embodiments of the invention. The unit 20 includes a conveyor belt that moves tablets 21 toward a bottling stage. The tablets 21 include the powder mixture. Other form factors may be added to the tablets 21. Finished tablets 21 (produced by the unit 20) are then forwarded to other parts of the manufacturing system, e.g., inspection units, packaging units, bottling units etc.

To the extent that the terms “including,” “includes,” “having,” “has,” “with,” or variants thereof are used in either the detailed description and the claims, such terms are intended to be inclusive in a similar manner to the term “comprising.”

Although the invention has been explained in relation to its preferred embodiment, it is to be understood that many other possible modifications and variations can be made without departing from the spirit and scope of the invention.

It will be appreciated by those skilled in the art that other various modifications could be made to the device without parting from the spirit and scope of this disclosure (especially various programmable features and architecture). All such modifications and changes fall within the scope of the claims and are intended to be covered thereby. The scope of the invention, therefore, is not intended to be limited to the exemplary embodiments and aspects that are shown and described herein. Rather, the scope and spirit of the invention is embodied by the appended claims.

What is claimed is:

1. A composition comprising:
curcumin; and
piperine combined together to produce a powder mixture that will counteract the effects of a hangover.

2. The composition of claim 1, wherein the powder mixture includes curcumin that is 95% curcuminoids and the powder mixture is blended with a liquid form resulting in a fast release effect.

3. The composition of claim 1, wherein the powder mixture includes curcumin that is 98% curcuminoids and in capsule form resulting in a slow-release effect.

4. The composition of claim 3, wherein the capsule includes a membrane that dissolves once ingested.

5. The composition of claim 4, wherein the membrane dissolves and the powder mixture is slowly released over a period of time.

6. The composition of claim 1, wherein the powder mixture includes curcumin that is 98% curcuminoids and in tablet form resulting in a slow-release effect.
